(19) United States
(12) Patent Application Publication
(10) Pub. No.: US 2025/0201387 A1
(43) Pub. Date: Jun. 19, 2025

(54) INFORMATION PROCESSING APPARATUS, INFORMATION PROCESSING METHOD, AND PROGRAM

(71) Applicant: SONY GROUP CORPORATION, TOKYO (JP)

(72) Inventors: SOTA SHOMAN, TOKYO (JP); SATOSHI OZAKI, TOKYO (JP); JUNJI OTSUKA, TOKYO (JP); SHO INAYOSHI, TOKYO (JP)

(21) Appl. No.: 18/838,845

(22) PCT Filed: Jan. 5, 2023
(86) PCT No.: PCT/JP2023/000043

§ 371 Date: Aug. 15, 2024

(30) Foreign Application Priority Data
Feb. 24, 2022 (JP) 2022-027062

(51) Int. Cl. G16H 30/40; G16H 30/20

(52) U.S. Cl. CPC G16H 30/40; G16H 30/20

(57) ABSTRACT

An information processing apparatus according to one embodiment of the present technology includes an acquisition unit, a behavior output unit, a basis output unit, a presentation unit, and a storage unit. The acquisition unit acquires a treatment image related to treatment. The behavior output unit outputs behavior information related to a behavior of a medical device related to the treatment, and a basis region indicating position information of a basis on which the behavior information is output, by inputting the treatment image to each of a plurality of recognizers. The basis output unit outputs basis information related to the basis by inputting the treatment image cropped on the basis of the basis region to a classifier. The presentation unit presents a plurality of pieces of the behavior information and a plurality of pieces of the basis information to the user. The storage unit stores input information input by the user on the basis of the behavior information and the basis information, as learning data of the plurality of recognizers and the classifier.

### TECHNICAL FIELD

The present technology relates to an information processing apparatus, an information processing method, and a program that are applicable to machine learning or the like.

### BACKGROUND ART

Patent Literature 1 describes a medical data processing apparatus that performs predetermined processing on medical data related to a subject to output medical diagnostic data and also to output standardized medical data based on medical data, which is standardized for machine learning without performing some or all of predetermined processing. Thus, the improvement in accuracy of machine learning is achieved (paragraphs [0036] to [0066] of the specification, FIGS. 2 and 6, etc. of Patent Literature 1).

### CITATION LIST

### Patent Literature

Patent Literature 1: Japanese Patent Application Laid-open No. 2020-203018

### DISCLOSURE OF INVENTION

### Technical Problem

In such machine learning using a medical support robot, there is a demand for a technique capable of achieving extraction of optimal learning data.

In view of the circumstances as described above, it is an object of the present technology to provide an information processing apparatus, an information processing method, and a program that are capable of achieving extraction of optimal learning data.

### Solution to Problem

In order to achieve the object described above, an information processing apparatus according to one embodiment of the present technology includes an acquisition unit, a behavior output unit, a basis output unit, a presentation unit, and a storage unit.

The acquisition unit acquires a treatment image related to treatment.

The behavior output unit outputs behavior information related to a behavior of a medical device related to the treatment, and a basis region indicating position information of a basis on which the behavior information is output, by inputting the treatment image to each of a plurality of recognizers.

The basis output unit outputs basis information related to the basis by inputting the treatment image cropped on the basis of the basis region to a classifier.

The presentation unit presents a plurality of pieces of the behavior information and a plurality of pieces of the basis information to the user.

The storage unit stores input information input by the user on the basis of the behavior information and the basis information, as learning data of the plurality of recognizers and the classifier.

In this information processing apparatus, the behavior information related to a behavior of a medical device related to the treatment and the basis region indicating position information of a basis on which the behavior information is output are output by inputting the treatment image to each of a plurality of recognizers, and the basis information related to the basis is output by inputting the treatment image cropped on the basis of the basis region to a classifier. A plurality of pieces of behavior information and a plurality of pieces of basis information are presented to the user. Input information based on the behavior information and the basis information is stored as learning data of the plurality of recognizers and the classifier. This makes it possible to extract optimal learning data.

The behavior information may include at least one of position information, movement information, or motion information of the medical device.

The basis information may include the basis and a cause of lowering an accuracy of the basis. In this case, the basis may include at least one of a surgical tool, a shaft of the surgical tool, or an organ. The cause may include at least one of smoke, dirt of the surgical tool, dirt of a lens, or occlusion.

The plurality of recognizers may include a first recognizer that performs offline learning, and a second recognizer that performs online learning. In this case, the first recognizer may output first behavior information and a first basis region. The second recognizer may output second behavior information and a second basis region.

The basis output unit may output first basis information and second basis information by inputting a first treatment image cropped on the basis of the first basis region and a second treatment image cropped on the basis of the second basis region to the classifier.

The presentation unit may present, to the user, a graphical user interface (GUI) in which the first behavior information, the second behavior information, the first basis information, and the second basis information can be recognized.

The input information may include at least one of a selection of the behavior information, a selection of the determination basis, an input of new behavior information different from the behavior information, an input of new basis information different from the basis information, or a new cropped treatment image different from the cropped treatment image.

If the first behavior information or the second behavior information is correct, the storage unit may store the first behavior information or the second behavior information that is selected by the user via the GUI, as learning data of the plurality of recognizers and the classifier.

If the first behavior information and the second behavior information are incorrect, the storage unit may store third behavior information as learning data of the plurality of recognizers and the classifier, the third behavior information being different from the first behavior information and the second behavior information that are input by the user via the GUI.

If the first basis information or the second basis information is correct, the storage unit may store the first basis information or the second basis information that is selected by the user via the GUI, as learning data of the plurality of recognizers and the classifier.

If the first behavior information and the second basis information are correct, the storage unit may store new basis information input by the user via the GUI, as learning data of the plurality of recognizers and the classifier.

If the first basis information and the second basis information are incorrect, the storage unit may store third basis information as learning data of the plurality of recognizers and the classifier, the third basis information being different from the first basis information and the second basis information that are input by the user via the GUI.

An information processing method according to one embodiment of the present technology is an information processing method that is executed by a computer system and includes: acquiring a treatment image related to treatment; outputting behavior information related to a behavior of a medical device related to the treatment, and a basis region indicating position information of a basis on which the behavior information is output, by inputting the treatment image to each of a plurality of recognizers; outputting basis information related to the basis by inputting the treatment image cropped on the basis of the basis region to a classifier; presenting a plurality of pieces of the behavior information and a plurality of pieces of the basis information to the user; and storing input information input by the user on the basis of the behavior information and the basis information, as learning data of the plurality of recognizers and the classifier.

A program according to one embodiment of the present technology causes a computer system to execute: acquiring a treatment image related to treatment; outputting behavior information related to a behavior of a medical device related to the treatment, and a basis region indicating position information of a basis on which the behavior information is output, by inputting the treatment image to each of a plurality of recognizers; outputting basis information related to the basis by inputting the treatment image cropped on the basis of the basis region to a classifier; presenting a plurality of pieces of the behavior information and a plurality of pieces of the basis information to the user; and storing input information input by the user on the basis of the behavior information and the basis information, as learning data of the plurality of recognizers and the classifier.

### BRIEF DESCRIPTION OF DRAWINGS

FIG. 1 is a block diagram showing a configuration example of an information processing apparatus according to the present technology.

FIG. 2 is a flowchart in learning of a behavior determiner.

FIG. 3 is a flowchart of selecting a recognition result.

FIG. 4 is a diagram schematically showing a treatment image, a basis region, and a cropped image.

FIG. 5 is a schematic diagram showing a cropped image and a determination basis.

FIG. 6 is a schematic diagram showing another example of a cropped image and a determination basis.

FIG. 7 is a schematic diagram showing another example of a cropped image and a determination basis.

FIG. 8 is a schematic diagram showing an example of a GUI in which a recognition result is displayed.

FIG. 9 is a schematic diagram showing an example of a GUI in which a recognition result is displayed.

FIG. 10 is a schematic diagram showing an example of a GUI when there are three or more behavior determiners.

FIG. 11 is a schematic diagram showing an example of a GUI in which a determination basis is displayed.

FIG. 12 is a schematic diagram showing an example of a GUI in which a determination basis is displayed when a recognition result is hindered.

FIG. 13 is a schematic diagram showing an example of a GUI when a recognition result and a determination basis are different.

FIG. 14 is a schematic diagram showing an GUI when the determination basis is incorrect.

FIG. 15 is a flowchart of learning of a determination basis classifier.

FIG. 16 is a block diagram showing a hardware configuration example of the information processing apparatus.

### MODE(S) FOR CARRYING OUT THE INVENTION

Hereinafter, an embodiment according to the present technology will be described with reference to the drawings.

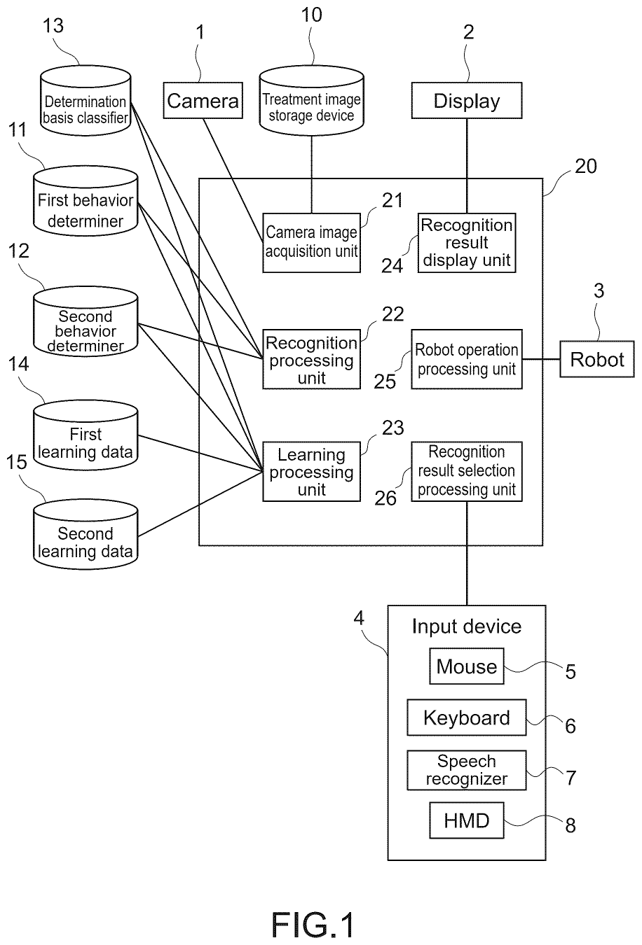

FIG. 1 is a block diagram showing a configuration example of an information processing apparatus 20 according to the present technology.

In this embodiment, the information processing apparatus 20 is communicably connected to a camera 1, a display 2, a robot 3, and an input device 4 via a predetermined communication net (network) by wires or wirelessly.

The camera 1 captures a treatment image related to treatment. For example, the treatment image includes an image in which an affected area or a medical scene (treatment process) such as a laparotomy for extracting an affected area is imaged. In addition to the above, the treatment image includes an image captured before treatment is performed, or when treatment is completed, such as after suturing is completed. In other words, a part where treatment is to be performed or a part where treatment has been performed is also included in the treatment image. Further, in addition to the part, an image including the whole of a patient may be included.

The robot 3 includes a medical device to be used in treatment. For example, the robot 3 includes a robot arm that operates (moves) the camera 1 and a control device that can control an imaging timing of the camera 1. In addition to the above, a laser apparatus for irradiating the retina or the like with a laser or a device for operating and controlling a surgical tool such as a scalpel may be included.

As shown in FIG. 1, the information processing apparatus 20 includes a camera image acquisition unit 21, a recognition processing unit 22, a learning processing unit 23, a recognition result display unit 24, a robot operation processing unit 25, and a recognition result selection processing unit 26.

The camera image acquisition unit 21 acquires a treatment image capture by the camera 1. In this embodiment, the camera image acquisition unit 21 includes a treatment image storage device 10 and stores a captured treatment image therein. Note that the treatment image may include a moving image.

The recognition processing unit 22 outputs a recognition result and a basis region on the basis of the treatment image. In this embodiment, the treatment image is input to each of a first behavior determiner 11 and a second behavior determiner 12, and thus the recognition processing unit 22 outputs a recognition result related to a behavior of a medical device related to treatment, and a basis region indicating position information of a basis on which behavior information is output.

The recognition result is information related to how to move the robot 3. For example, the recognition result includes position information and movement information indicating which region a user (medical personnel) should focus on and move to from now on. For example, on the basis of a direction and a distance in which the user moves a surgical tool, a position and a direction in which the camera 1 is to be moved are output as a recognition result. Further, the recognition result may include a motion related to the robot 3, such as a position and a timing at which a laser is applied and control of an imaging magnification. Note that, in this embodiment, the recognition result corresponds to behavior information related to the behavior of the medical device related to the treatment.

The basis region indicates a basis that is a referenced part of the treatment image to derive a recognition result. For example, each pixel of the treatment image contains a value. As the value becomes higher, the behavior determiner is more likely to use it as the basis of the recognition result. Further, in this embodiment, an explainable AI (XAI) is used to output which region of the treatment image is focused on, that is, a basis region as a region where the basis on which the recognition result is output seems to exist.

Further, the recognition processing unit 22 crops a corresponding portion on the basis of the output basis region. When the cropped treatment image is input to a determination basis classifier 13, a determination basis is output. The determination basis classifier 13 is an AI for classifying the determination basis of each behavior determiner.

The determination basis indicates a basis on which the recognition result output by the behavior determiner is output. For example, the determination basis includes: a basis on which a recognition result such as a surgical tool, a shaft of a surgical tool, or an organ is output; and a cause of an erroneous recognition in which a recognition result such as smoke, dirt of a surgical tool, dirt of a lens, or occlusion is not correctly output. Note that, in this embodiment, the determination basis corresponds to basis information related to the basis on which behavior information is output.

Hereinafter, the recognition result output from the first behavior determiner 11, and the basis region will be respectively referred to as a first recognition result and a first basis region. Further, the determination basis that is output by inputting a cropped treatment image to the determination basis classifier 13 on the basis of the first basis region will be referred to as a first determination basis. Similarly, for the second behavior determiner 12 as well, a second recognition result, a second basis region, and a second determination basis will be used.

In this embodiment, the first behavior determiner 11 is an AI for determining the behavior of the robot 3 by offline learning. Further, the second behavior determiner 12 is an AI for determining the behavior of the robot 3 by online learning.

Further, in this embodiment, the first behavior determiner 11 is trained by first learning data 14 obtained in advance. The second behavior determiner 12 is trained by second learning data 15 obtained during or after the treatment. Note that there may be a plurality of pieces of first learning data 14. Further, there may be a total of three or more pieces of learning data and behavior determiners. In addition, each behavior determiner may be trained by any one of offline learning or online learning. Note that there are as many learning models as the number of pieces of learning data.

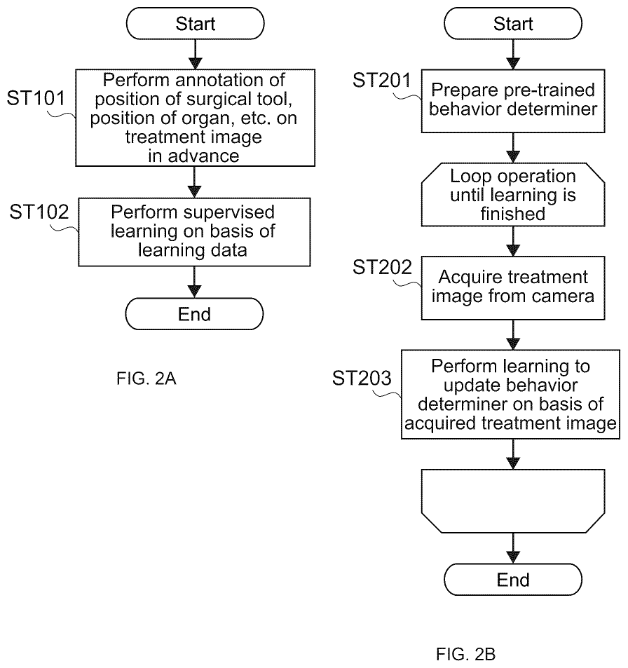

FIG. 2 is a flowchart in learning of the behavior determiner. A of FIG. 2 is a flowchart of offline learning. B of FIG. 2 is a flowchart of online learning.

As shown in A of FIG. 2, the first behavior determiner 11 is trained as follows. Annotation of the position of a surgical tool, the position of an organ, or the like is performed on the treatment image in advance (Step 101). Further, supervised learning is performed on the basis of the learning data (Step 102).

In this embodiment, information related to the position and type of the surgical tool or the organ, diagnosis information of a patient, and the like are used as teacher data of offline learning. For example, a positional relationship of organs, a surgical tool used for an organ, a disease name, the position and type of an affected area related to the disease name, and the like may be used.

As shown in B of FIG. 2, the second behavior determiner 12 is trained as follows. A pre-trained behavior determiner is prepared (Step 201). Treatment is acquired from the camera 1 (Step 202). The behavior determiner is trained and updated on the basis of the acquired treatment image (Step 203). Steps 202 and 203 are looped until the learning is finished.

In this embodiment, in online learning, learning is sequentially performed on the basis of the treatment images obtained from the camera 1, and a parameter of the behavior determiner is updated. For teacher data, an AI for generating a label, such as a quasi-label generator trained in advance, may be used, or unsupervised learning may be performed.

Referring back to FIG. 1, the learning processing unit 23 performs learning of the first behavior determiner 11, the second behavior determiner 12, and the determination basis classifier 13. In this embodiment, the learning processing unit 23 performs learning from input information input by the user, which is based on the recognition result and the determination basis presented to the user.

The recognition result display unit 24 presents the recognition result and the determination basis output from the recognition processing unit 22. In this embodiment, the recognition result display unit 24 displays, on the display 2, a graphical user interface (GUI) in which a first recognition result, a second recognition result, a first determination basis, and a second determination basis can be visually recognized.

In this embodiment, the user selects a recognition result via the presented GUI by using the input device 4. For example, the user may select a recognition result by using a mouse 5. In addition to the above, the user may select a recognition result displayed on the display 2 by touching the recognition result.

Further, the user can also correct the presented determination basis. For example, a keyboard 6 may be used to input a correct determination basis. In addition to the above, the recognition result may be selected, and the determination basis may be corrected, by speech recognition 7 (e.g., microphone). Further, in addition to the display 2, a head mounted display (HMD) 8 may be used for the GUI.

In other words, the input information includes at least one of selection of a recognition result and a determination basis, or correction of a recognition result and a determination basis (input of a correct recognition result and a correct determination basis). Further, the user can input the input information via the GUI. In addition to the above, various types of information input by the user via the GUI may be included. Specific selection method and correction method will be described later.

The robot operation processing unit 25 executes the operation processing related to the robot 3. For example, the operation processing includes output of a signal for controlling an imaging timing of the camera 1, output of a signal for driving a robot arm that supports the camera 1, and the like.

The recognition result selection processing unit 26 processes the recognition result selected via the GUI presented by the recognition result display unit 24. In this embodiment, the recognition result selection processing unit 26 stores the selected recognition result and the determination basis in the recognition result (or the corrected determination basis) in a storage area.

Note that, in this embodiment, the camera image acquisition unit 21 corresponds to an acquisition unit that acquires a treatment image related to treatment.

Note that, in this embodiment, the recognition processing unit 22 functions as: a behavior output unit that outputs behavior information related to a behavior of a medical device related to the treatment, and a basis region indicating position information of a basis on which the behavior information is output, by inputting the treatment image to each of a plurality of recognizers; and a basis output unit that outputs basis information related to the basis by inputting the treatment image cropped on the basis of the basis region to a classifier.

Note that, in this embodiment, the recognition result display unit 24 corresponds to a presentation unit that presents a plurality of pieces of behavior information and a plurality of pieces of basis information to the user.

Note that, in this embodiment, the recognition result selection processing unit 26 corresponds to a storage unit that stores the input information that is input by the user and based on the behavior information and the basis information, as learning data of the plurality of recognizers and the classifier.

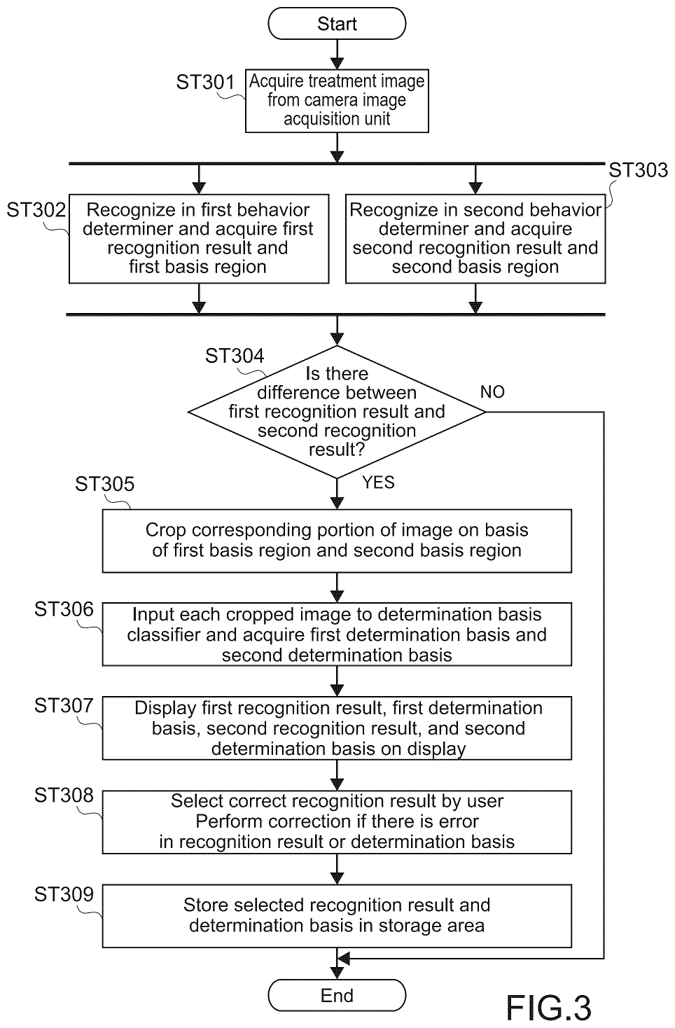

FIG. 3 is a flowchart of selecting a recognition result.

As shown in FIG. 3, the camera image acquisition unit 21 acquires a treatment image (Step 301).

The recognition processing unit 22 acquires a first recognition result and a first basis region that have been output by inputting the treatment image to the first behavior determiner 11 (Step 302). Similarly, the recognition processing unit 22 acquires a second recognition result and a second basis region that have been output by inputting the treatment image to the second behavior determiner 12 (Step 303). Note that in FIG. 3, Step 302 and Step 303 are processed in parallel. The present technology is not limited to the above, and Step 302 and Step 303 may be processed in series.

The recognition processing unit 22 determines whether or not there is a difference between the first recognition result and the second recognition result (Step 304). For example, it is determined whether or not the position indicated by the recognition result, the direction of movement (the amount of movement), and the like are different therebetween.

If there is a difference between the recognition results (YES in Step 304), a corresponding portion of the treatment image is cropped on the basis of the basis regions (Step 305). Hereinafter, the cropped treatment image will be referred to as a cropped image.

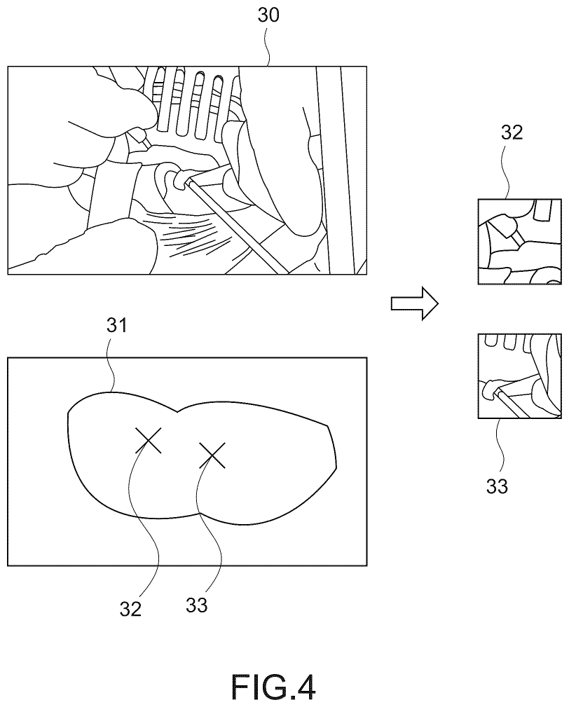

FIG. 4 is a diagram schematically showing the treatment image, the basis region, and the cropped image.

As shown in FIG. 4, a treatment image 30 is input to the first behavior determiner 11 or the second behavior determiner 12, and thus a basis region 31 is output. Note that, in FIG. 4, the basis region 31 is described as a region for the purpose of description, but actually contains a numerical value indicating how much the recognition result is influenced for each pixel.

In this embodiment, a rectangular region centering on the range having a local maximal value of each pixel value in the basis region 31 is cropped, so that a cropped image is acquired. For example, in FIG. 4, a mark 32 and a mark 33 in the basis region 31 indicate pixels each having a local maximal value. In other words, the example of FIG. 4 indicates that two local maximal values are obtained, and a rectangular region centering on each local maximal value is a range to be cropped. Note that at least one or more cropped images exist for a single recognition result. Further, as in the example of FIG. 4, two or more cropped images may exist.

A first cropped image based on the first basis region and a second cropped image based on the second basis region are each input to the determination basis classifier 13, and thus a first determination basis and a second determination basis are acquired (Step 306).

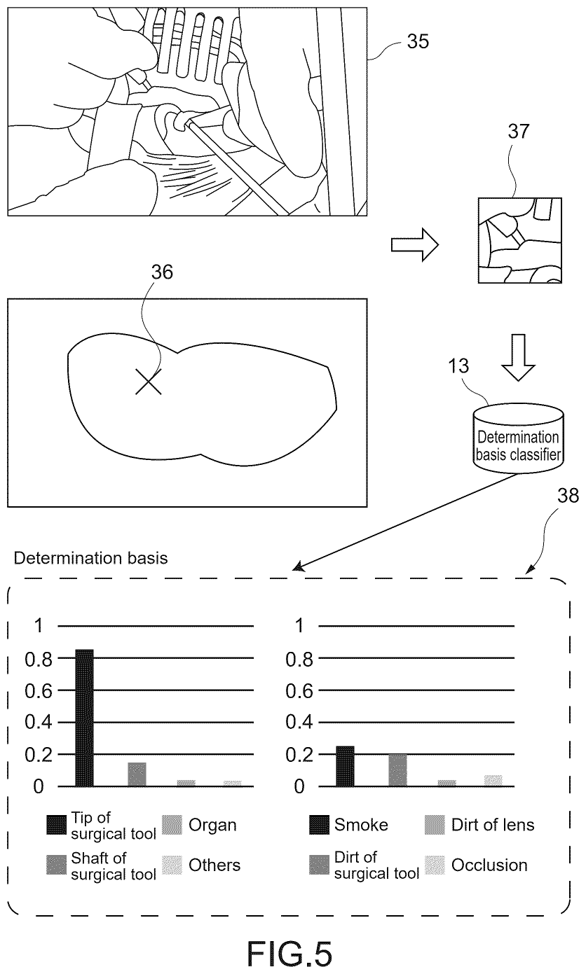

FIG. 5 is a schematic diagram showing a cropped image and a determination basis.

As shown in FIG. 5, a cropped image 37 is acquired on the basis of a treatment image 35 and a basis region 36. The cropped image 37 is input to the determination basis classifier 13, and thus a determination basis 38 is output.

In FIG. 5, a graph shown on the left indicates what objects appear in the cropped region. As shown in the graph, candidates of the objects appearing in the region include a tip of a surgical tool, a shaft of a surgical tool, an organ, and the like, and an item having a high numerical value is high likely to be an object appearing in the region.

Further, a graph shown on the right indicates whether or not a cause assumed to be a cause of an erroneous recognition in treatment is included. For example, candidates of the cause include smoke, dirt of a surgical tool, dirt of a lens, and occlusion. Of those candidates, an item having a high numerical value is highly likely to be a cause of an erroneous recognition.

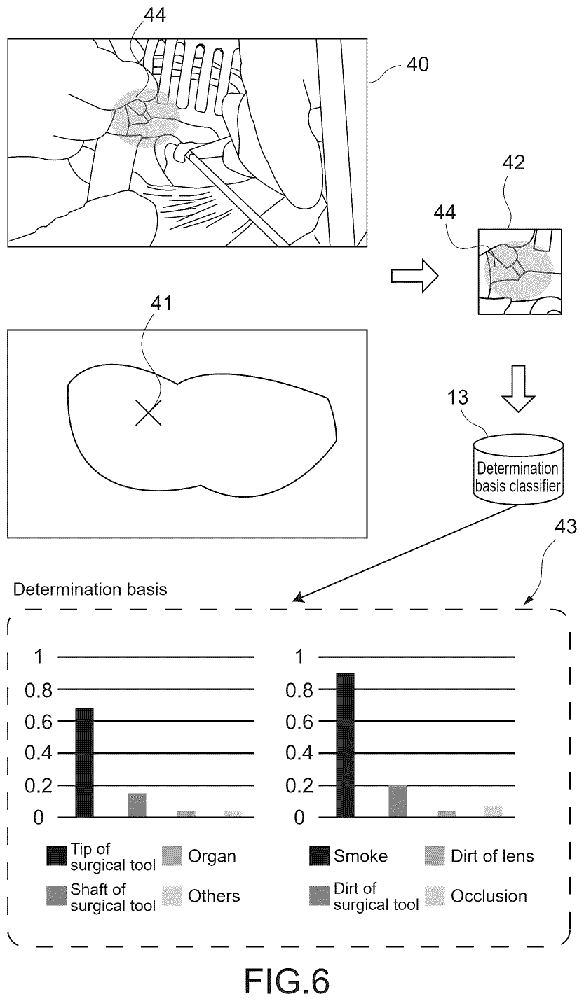

FIG. 6 is a schematic diagram showing another example of a cropped image and a determination basis.

Also in FIG. 6, similarly to FIG. 5, a cropped image 42 is acquired on the basis of a treatment image 40 and a mark (pixel having a local maximal value) 41 of the basis region. The cropped image 42 is input to the determination basis classifier 13, and thus a determination basis 43 is output.

As shown in FIG. 6, the cropped image 42 include smoke 44. In this case, a graph shown on the right indicates the smoke 44 with a high numerical value because the smoke 44 is highly likely to be a cause of an erroneous recognition. Further, due to the smoke 44, an object appearing in the cropped image 42 is less likely to be the tip of a surgical tool, that is, a low numerical value is displayed.

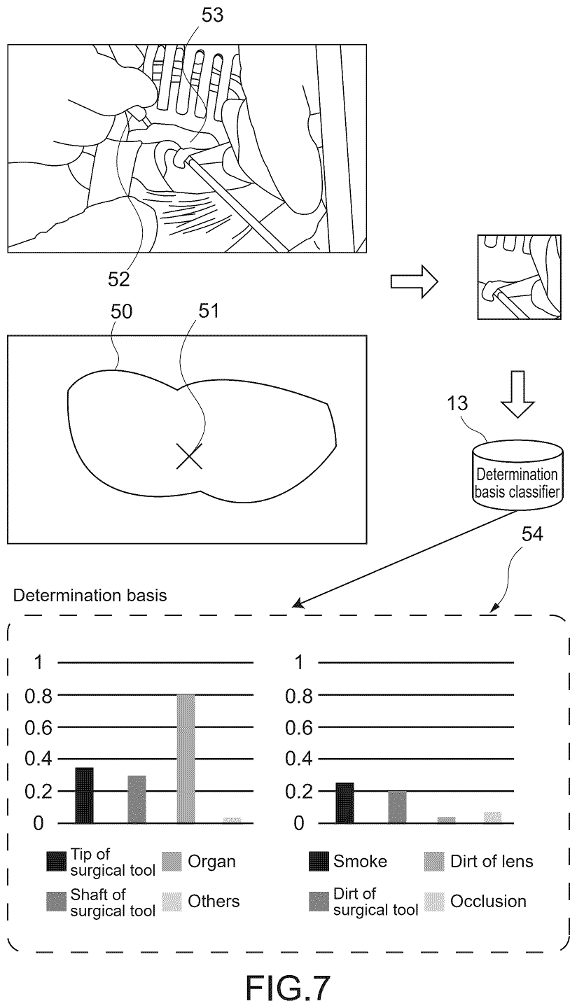

FIG. 7 is a schematic diagram showing another example of a cropped image and a determination basis.

In FIG. 7, a mark 51 of a basis region 50 indicates not a surgical tool 52 but an organ 53. The determination basis classifier 13 sets the organ as a determination basis for output when a cropped image is input (see a graph 54).

In this case, if it is known in advance that a specific organ is important, the determination basis classifier 13 is trained so as to be capable of determining whether or not it is an organ as shown in the graph 54. For example, if a patient has appendicitis, the procedure of the operation for removing the appendicitis, the position and shape of the appendicitis, and the like are trained.

Referring back to FIG. 3, the recognition result display unit 24 displays, on the display 2, a GUI in which a first recognition result, a first determination basis, a second recognition result, and a second determination basis can be visually recognized (Step 307).

If a correct recognition result is selected by the user or if there is an error in the recognition result or the determination basis, a corrected recognition result or determination basis is input (Step 308). The selected recognition result and determination basis (or the corrected recognition result and determination basis) are stored in the storage area (Step 309).

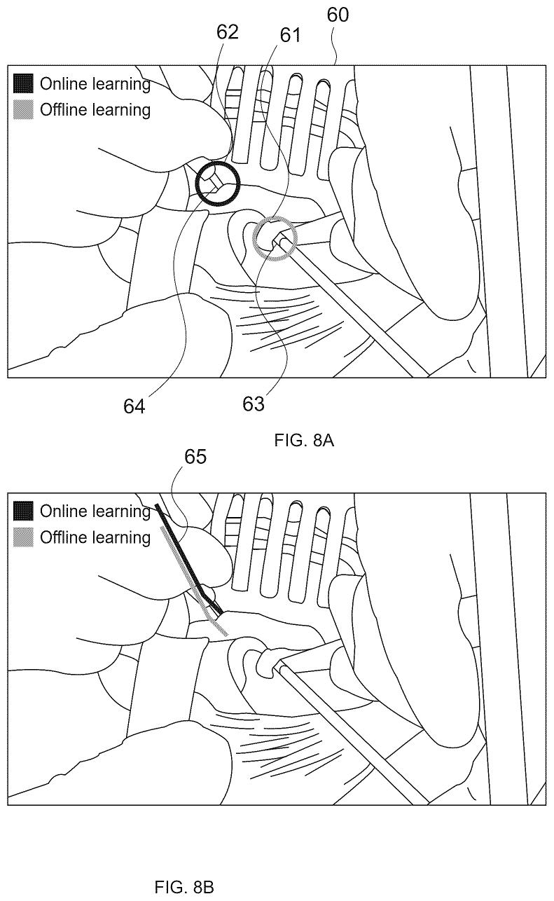

FIG. 8 is a schematic diagram showing an example of a GUI in which a recognition result is displayed. A of FIG. 8 is a schematic diagram showing an example of a method of displaying a recognition result. B of FIG. 8 is a schematic diagram showing another example of a method of displaying a recognition result.

As shown in A of FIG. 8, the recognition result display unit 24 displays, on the display 2, a GUI in which a first recognition result 61 output from the first behavior determiner 11, and a second recognition result 62 output from the second behavior determiner 12 can be recognized in a treatment image 60.

For example, in A of FIG. 8, two recognition results are shown assuming that regions located ahead of the tips of a surgical tool 63 and a surgical tool 64 different from each other are regarded as important. The user selects a correct recognition result from the displayed recognition results.

Note that the method of displaying a recognition result is not limited, and as shown in B of FIG. 8, the skeleton of a surgical tool 65 may be displayed as a recognition result.

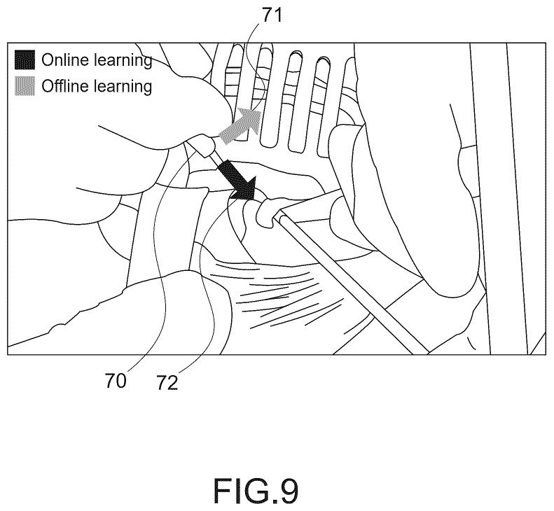

FIG. 9 is a schematic diagram showing an example of a GUI in which a recognition result is displayed.

As shown in FIG. 9, movement information may be displayed in addition to the position information as a recognition result. For example, in FIG. 9, a recognition result 71 and a recognition result 72 indicating a direction in which a surgical tool 70 moves are displayed.

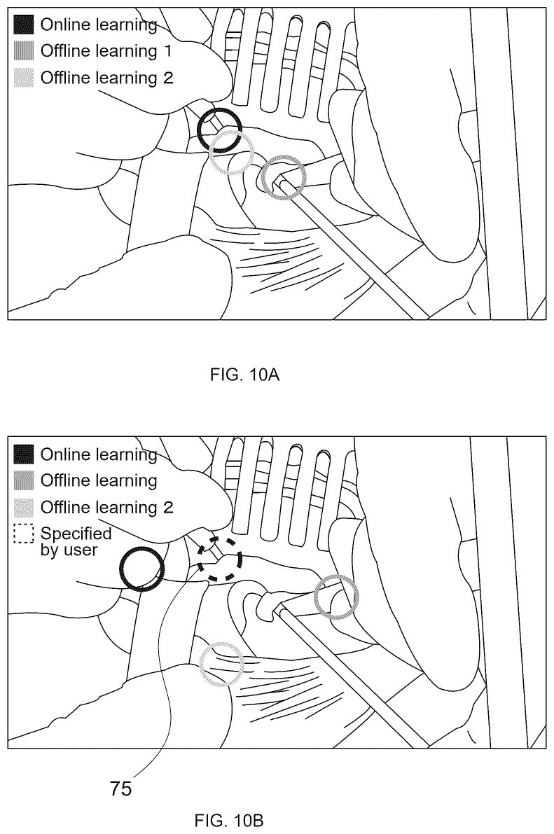

FIG. 10 is a schematic diagram showing an example of a GUI when there are three or more behavior determiners. A of FIG. 10 is a schematic diagram showing an example of a GUI when there are three recognition results.

FIG. 10 shows a GUI when a third behavior determiner exists, the third behavior determiner being different from the first behavior determiner 11 subjected to online learning and the second behavior determiner 12 subjected to offline learning. For example, in A of FIG. 10, the three recognition results indicate results obtained by estimating a region of interest of a doctor (user).

As shown in FIG. 10, the third behavior determiner is a behavior determiner subjected to offline learning (offline learning 2 shown in FIG. 10). The present technology is not limited to the above, and online learning may be used, and the number of behavior determiners and a learning method are not limited.

The user selects the displayed recognition result. If there are three or more recognition results, two or more recognition results may be selected. If two or more recognition results are selected, all of the recognition results are stored in the storage area.

B of FIG. 10 is a schematic diagram showing an example of a GUI when all of the displayed recognition results are incorrect.

As shown in B of FIG. 10, if all of the three recognition results output from the behavior determiners are incorrect, the user specifies a correct position (broken line 75). The specified position (input information) is stored as new teacher data in the storage area.

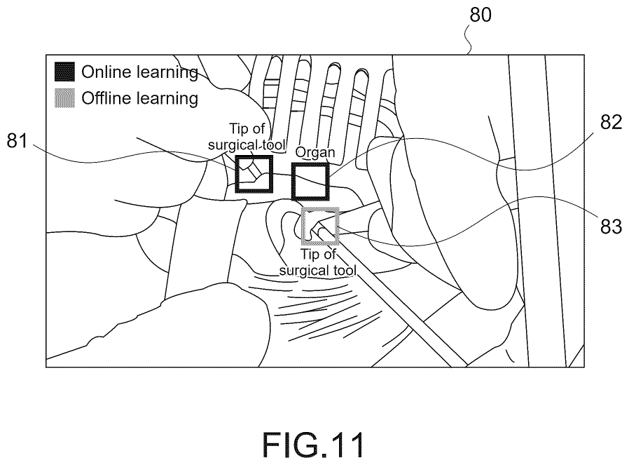

FIG. 11 is a schematic diagram showing an example of a GUI in which a determination basis is displayed.

In FIG. 11, the recognition result display unit 24 presents a GUI 80 in which a determination basis can be recognized. As shown in FIG. 11, regions in the cropped image surrounded with rectangular shapes (black frames 81 and 82 and gray frame 83) are displayed, and determination bases are displayed on those rectangular shapes.

For example, if the recognition results (gray circle 61 and black circle 62) are output as shown in FIG. 8, a determination basis having the highest numerical value in the determination basis classifier 13, which corresponds to the recognition result (see FIG. 5), is displayed. This makes it easier for the user to select a correct recognition result.

Note that a plurality of regions in the cropped image may be displayed for one recognition result.

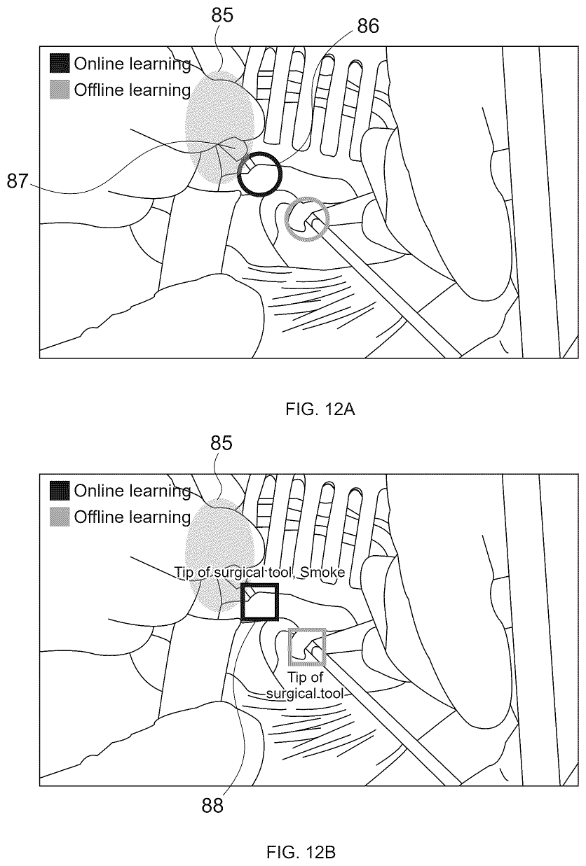

FIG. 12 is a schematic diagram showing an example of a GUI in which a determination basis is displayed when a recognition result is hindered.

A of FIG. 12 is a schematic diagram showing a GUI in which a recognition result can be visually recognized. As shown in A of FIG. 12, smoke 85 occurs, and thus the position of a second recognition result 86 output from the second behavior determiner 12 is displayed with a displacement from a surgical tool 87.

B of FIG. 12 is a schematic diagram showing a GUI in which a determination basis can be visually recognized. As shown in B of FIG. 12, smoke 85 occurs, and thus a position of a region 88 in the cropped image is displaced. Further, the smoke 85 appearing in the region of the cropped image is displayed. In other words, “tip of surgical tool, smoke” is displayed as a determination basis on the region 88, so that the user can easily understand why an erroneous recognition result is output.

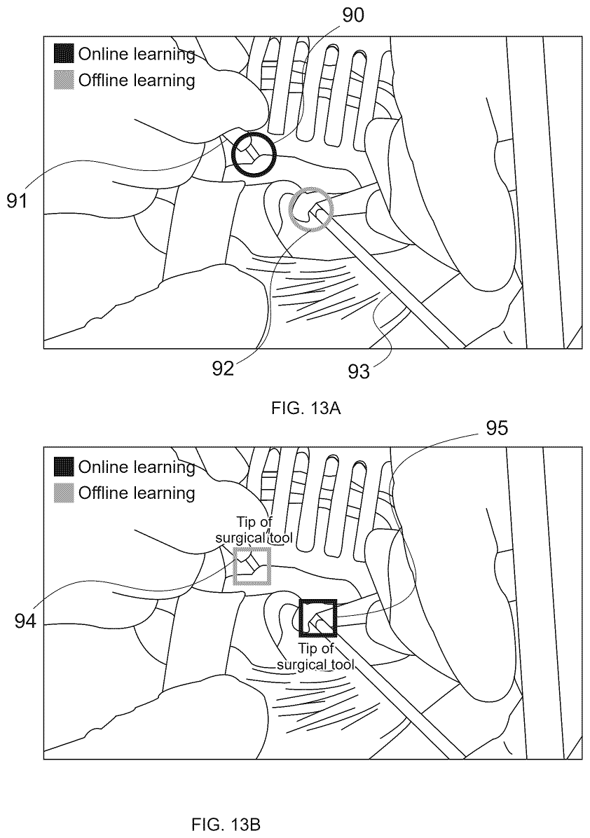

FIG. 13 is a schematic diagram showing an example of a GUI when a recognition result and a determination basis are different. A of FIG. 13 is a schematic diagram showing a GUI in which a recognition result can be visually recognized. B of FIG. 13 is a schematic diagram showing a GUI in which a determination basis can be visually recognized.

As shown in A of FIG. 13, a second recognition result 90 output from the second behavior determiner 12 is displayed at a position indicating the tip of a surgical tool 91. Further, a first recognition result 92 output from the first behavior determiner 11 is displayed at a position indicating the tip of a surgical tool 93.

Further, as shown in B of FIG. 13, a first determination basis 94 is displayed at a position at which the second recognition result 90 is displayed. Further, a second determination basis 95 is displayed at a position at which the first recognition result 92 is displayed.

In FIG. 13, it is assumed that the second recognition result 90 and the first determination basis 94 are correct. In other words, if the second determination basis 92 for the second recognition result 90 is incorrect, the user corrects the second determination basis 92 and teaches a correct region in the cropped image and a correct determination basis. The correct region and determination basis presented by the user are stored in the storage area, and are utilized at the time of the next learning of the determination basis classifier 13.

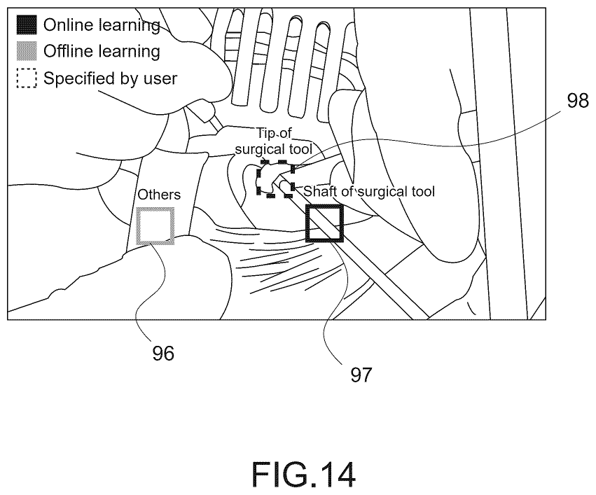

FIG. 14 is a schematic diagram showing an GUI when a determination basis is incorrect.

As shown in FIG. 14, if a first determination basis 96 and a second determination basis 97 are incorrect, or if there are a region of interest in the cropped image and a determination basis of interest other than the first determination basis 96 and the second determination basis 97, the user teaches a correct region (broken line 98) in the cropped image and a correct determination basis (tip of surgical tool).

Further, the user teaches a correct determination basis similarly if the position of the region in the cropped image is correct and the determination basis is incorrect.

As described above, the information processing apparatus 20 according to this embodiment acquires a treatment image related to treatment. The treatment image is input to each of the first behavior determiner 11 and the second behavior determiner 12, and thus a recognition result related to the behavior of a medical device related to the treatment, and a basis region indicating the position information of a basis on which the recognition result is output are output. The treatment image cropped on the basis of the basis region is input to the determination basis classifier 13, so that a determination basis is output. A first recognition result and a second recognition result, and first basis information and second basis information are presented to the user. The recognition result and determination basis selected by the user are stored as learning data of the first behavior determiner 11, the second behavior determiner 12, and the determination basis classifier 13. This makes it possible to extract optimal learning data.

Conventionally, a medical support robot has performed medical support on the basis of a model trained using a database collected in advance, but it has been difficult to achieve a motion of the robot in accordance with conditions in actual use. Further, in a diagnostic support AI, it is difficult to pursue what kind of learning data should be used for learning such that a robot performs an optimal behavior in accordance with a doctor or an environment.

In the present technology, if there is a difference between recognition results, the recognition results and the determination bases thereof are respectively displayed, so that the user can determine which one is correct or determines whether both of them are incorrect and need correct teaching, and can perform correction thereof. The corrected information is stored in the storage area and is utilized at the time of the next learning.

In other words, the behavior of the robot by the model trained using an offline database, and the behavior of the robot by the model trained using an online database are determined by a medical professional, so that the training data resulting from an excellent behavior is extracted, and a new model is trained using a sophisticated database. Further, the determination basis is displayed together, which makes easier for a medical professional to perform determination.

### Other Embodiments

The present technology is not limited to the embodiment described above, and can implement various other embodiments.

In the embodiment described above, the treatment image is cropped, and the user selects or corrects the determination basis and the recognition result of that treatment image. The present technology is not limited to the above, and similar processing may be performed on a moving image. For example, while a moving image in which treatment is performed is being reproduced, a recognition result and a determination basis may be displayed in assimilation, and selection or correction may be performed by the user.

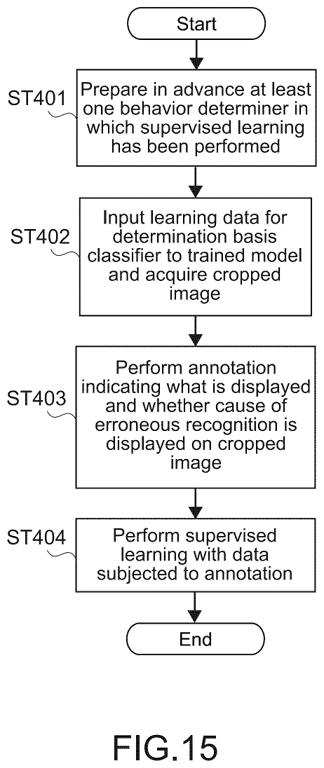

FIG. 15 is a flowchart of learning of the determination basis classifier 13.

As shown in FIG. 15, the determination basis classifier 13 is trained as follows. For preparation, at least one behavior determiner in which supervised learning has been performed is prepared in advance (Step 401). Learning data for the determination basis classifier is input to a trained model, and a cropped image is acquired by the method similar to that of FIG. 3 (Step 402). Annotation indicating what is displayed and whether a cause of an erroneous recognition is displayed is performed on the acquired cropped image (Step 403). Supervised learning is performed with the data subjected to the annotation (Step 405).

The determination basis classifier 13 is trained in advance in the steps described above. Further, if the determination basis has an error and the user directly corrects the error, the determination basis classifier 13 is trained again on the basis of the taught data.

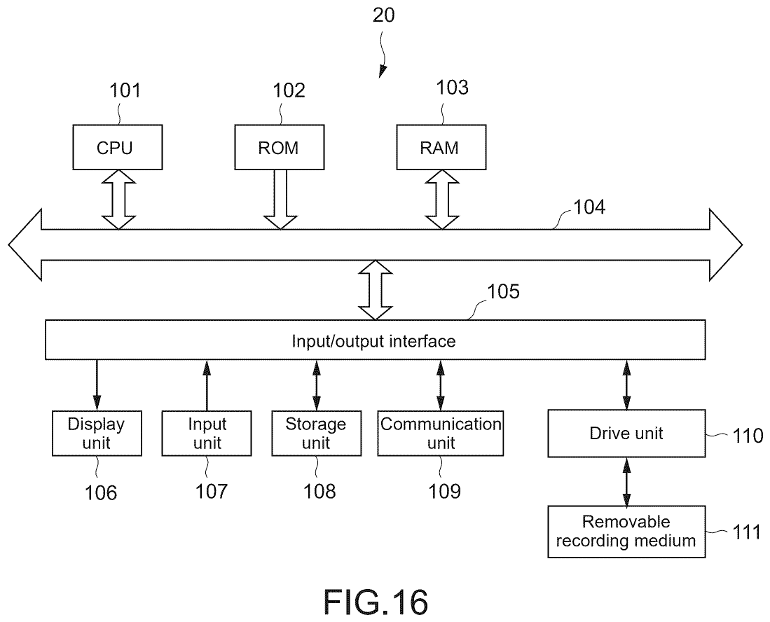

FIG. 16 is a block diagram showing a hardware configuration example of the information processing apparatus 20.

The information processing apparatus 20 includes a central processing unit (CPU) 101, a read-only memory (ROM) 102, a random-access memory (RAM) 103, an input/output interface 105, and a bus 104 that connects those components to each other. A display unit 106, an input unit 107, a storage unit 108, a communication unit 109, a drive unit 110, and the like are connected to the input/output interface 105.

The display unit 106 is, for example, a display device using liquid crystal, electro-luminescence (EL), or the like. The input unit 107 is, for example, a keyboard, a pointing device, a touch panel, or another operation device. If the input unit 107 includes a touch panel, the touch panel may be integrated with the display unit 106.

The storage unit 108 is a nonvolatile storage device and is, for example, a hard disk drive (HDD), a flash memory, or another solid-state memory. The drive unit 110 is, for example, a device capable of driving a removable recording medium 111 such as an optical recording medium or a magnetic recording tape.

The communication unit 109 is a modem, a router, or another communication device that can be connected to a local area network (LAN), a wide area network (WAN), or the like for communicating with other devices. The communication unit 109 may communicate using wires or radios. The communication unit 109 is often used separately from the information processing apparatus 20.

The information processing by the information processing apparatus 20 having the hardware configuration as described above is implemented in cooperation with the software stored in the storage unit 108, the ROM 102, or the like, and the hardware resource of the information processing apparatus 20. Specifically, the information processing method according to the present technology is implemented when a program stored in the ROM 102 or the like and configuring the software is loaded to the RAM 103 and then executed.

The program is installed in the information processing apparatus 20, for example, through the recording medium 111. Alternatively, the program may be installed in the information processing apparatus 20 via a global network or the like. In addition, any non-transitory computer-readable storage medium may be used.

The information processing method and the program according to the present technology may be executed, and the evaluation unit according to the present technology may be constructed, by linking a computer mounted on a communication terminal with another computer capable of communicating via a network or the like.

In other words, the information processing apparatus, the information processing method, and the program according to the present technology can be executed not only in a computer system including a single computer but also in a computer system in which a plurality of computers operates in conjunction with each other. Note that, in the present disclosure, a system means a collection of a plurality of constituent elements (apparatuses, modules (components), and the like), and whether or not all the constituent elements are in the same housing is not limited. Therefore, a plurality of apparatuses accommodated in separate housings and connected to each other through a network, and a single apparatus in which a plurality of modules is accommodated in a single housing are both the system.

The execution of the information processing apparatus, the information processing method, and the program according to the present technology by a computer system includes, for example, both a case where the output of a recognition result, the output of a basis region, the output of a determination basis, and the like are executed by a single computer and a case where each process is executed by a different computer. Further, the execution of each process by a predetermined computer includes causing another computer to execute a part or all of the processes and acquiring a result thereof.

In other words, the information processing apparatus, the information processing method, and the program according to the present technology are also applicable to a configuration of cloud computing in which a single function is shared and cooperatively processed by a plurality of apparatuses through a network.

The configurations of the recognition processing unit, the learning processing unit, the recognition result display unit, and the like; the control flow of the communication system; and the like described with reference to the respective figures are merely embodiments, and any modifications may be made thereto without departing from the spirit of the present technology. In other words, any other configurations or algorithms for the purpose of practicing the present technology may be adopted.

Note that the effects described in the present disclosure are not limitative but are merely illustrative, and other effects may be provided. The description on the plurality of effects does not mean that those effects are not necessarily exerted at the same time. It means that at least any of the effects described above is obtained depending on conditions or the like, and as a matter of course, effects not described in the present disclosure may be exerted.

At least two of the characteristic portions according to each embodiment described above can be combined. In other words, the various characteristic portions described in each embodiment may be discretionarily combined without distinguishing between the embodiments.

Note that the present technology may also take the following configurations.

(1) An information processing apparatus, including:
an acquisition unit that acquires a treatment image related to treatment;
a behavior output unit that outputs behavior information related to a behavior of a medical device related to the treatment, and a basis region indicating position information of a basis on which the behavior information is output, by inputting the treatment image to each of a plurality of recognizers;
a basis output unit that outputs basis information related to the basis by inputting the treatment image cropped on the basis of the basis region to a classifier;
a presentation unit that presents a plurality of pieces of the behavior information and a plurality of pieces of the basis information to the user; and
a storage unit that stores input information input by the user on the basis of the behavior information and the basis information, as learning data of the plurality of recognizers and the classifier.
   (2) The information processing apparatus according to (1), in which
the behavior information includes at least one of position information, movement information, or motion information of the medical device.
   (3) The information processing apparatus according to (1), in which
the basis information includes the basis and a cause of lowering an accuracy of the basis,
the basis includes at least one of a surgical tool, a shaft of the surgical tool, or an organ, and
the cause includes at least one of smoke, dirt of the surgical tool, dirt of a lens, or occlusion.
   (4) The information processing apparatus according to (1), in which
the plurality of recognizers includes
   a first recognizer that performs offline learning, and
   a second recognizer that performs online learning,
the first recognizer outputs first behavior information and a first basis region, and
the second recognizer outputs second behavior information and a second basis region.
   (5) The information processing apparatus according to (4), in which
the basis output unit outputs first basis information and second basis information by inputting a first treatment image cropped on the basis of the first basis region and a second treatment image cropped on the basis of the second basis region to the classifier.
   (6) The information processing apparatus according to (5), in which
the presentation unit presents, to the user, a graphical user interface (GUI) in which the first behavior information, the second behavior information, the first basis information, and the second basis information can be recognized.
   (7) The information processing apparatus according to (6), in which
the input information includes at least one of a selection of the behavior information, a selection of the determination basis, an input of new behavior information different from the behavior information, an input of new basis information different from the basis information, or a new cropped treatment image different from the cropped treatment image.
   (8) The information processing apparatus according to (7), in which
if the first behavior information or the second behavior information is correct, the storage unit stores the first behavior information or the second behavior information that is selected by the user via the GUI, as learning data of the plurality of recognizers and the classifier.
   (9) The information processing apparatus according to (7), in which
if the first behavior information and the second behavior information are incorrect, the storage unit stores third behavior information as learning data of the plurality of recognizers and the classifier, the third behavior information being different from the first behavior information and the second behavior information that are input by the user via the GUI.
   (10) The information processing apparatus according to (7), in which
if the first basis information or the second basis information is correct, the storage unit stores the first basis information or the second basis information that is selected by the user via the GUI, as learning data of the plurality of recognizers and the classifier.
   (11) The information processing apparatus according to (7), in which
if the first behavior information and the second basis information are correct, the storage unit stores new basis information input by the user via the GUI, as learning data of the plurality of recognizers and the classifier.
   (12) The information processing apparatus according to (7), in which
if the first basis information and the second basis information are incorrect, the storage unit stores third basis information as learning data of the plurality of recognizers and the classifier, the third basis information being different from the first basis information and the second basis information that are input by the user via the GUI.
   (13) An information processing method that is executed by a computer system, including:
acquiring a treatment image related to treatment;
outputting behavior information related to a behavior of a medical device related to the treatment, and a basis region indicating position information of a basis on which the behavior information is output, by inputting the treatment image to each of a plurality of recognizers;
outputting basis information related to the basis by inputting the treatment image cropped on the basis of the basis region to a classifier;
presenting a plurality of pieces of the behavior information and a plurality of pieces of the basis information to the user; and
storing input information input by the user on the basis of the behavior information and the basis information, as learning data of the plurality of recognizers and the classifier.
   (14) A program that causes a computer system to execute:
acquiring a treatment image related to treatment;
outputting behavior information related to a behavior of a medical device related to the treatment, and a basis region indicating position information of a basis on which the behavior information is output, by inputting the treatment image to each of a plurality of recognizers;
outputting basis information related to the basis by inputting the treatment image cropped on the basis of the basis region to a classifier;
presenting a plurality of pieces of the behavior information and a plurality of pieces of the basis information to the user; and
storing input information input by the user on the basis of the behavior information and the basis information, as learning data of the plurality of recognizers and the classifier.

### REFERENCE SIGNS LIST

11 first behavior determiner
12 second behavior determiner
13 determination basis classifier
20 information processing apparatus
21 camera image acquisition unit
22 recognition processing unit
23 learning processing unit
24 recognition result display unit
26 recognition result selection processing unit

1. An information processing apparatus, comprising:
an acquisition unit that acquires a treatment image related to treatment;
a behavior output unit that outputs behavior information related to a behavior of a medical device related to the treatment, and a basis region indicating position information of a basis on which the behavior information is output, by inputting the treatment image to each of a plurality of recognizers;
a basis output unit that outputs basis information related to the basis by inputting the treatment image cropped on the basis of the basis region to a classifier;
a presentation unit that presents a plurality of pieces of the behavior information and a plurality of pieces of the basis information to the user; and
a storage unit that stores input information input by the user on a basis of the behavior information and the basis information, as learning data of the plurality of recognizers and the classifier.

2. The information processing apparatus according to claim 1, wherein
the behavior information includes at least one of position information, movement information, or motion information of the medical device.

3. The information processing apparatus according to claim 1, wherein
the basis information includes the basis and a cause of lowering an accuracy of the basis,
the basis includes at least one of a surgical tool, a shaft of the surgical tool, or an organ, and
the cause includes at least one of smoke, dirt of the surgical tool, dirt of a lens, or occlusion.

4. The information processing apparatus according to claim 1, wherein
the plurality of recognizers includes
a first recognizer that performs offline learning, and
a second recognizer that performs online learning,
the first recognizer outputs first behavior information and a first basis region, and
the second recognizer outputs second behavior information and a second basis region.

5. The information processing apparatus according to claim 4, wherein
the basis output unit outputs first basis information and second basis information by inputting a first treatment image cropped on a basis of the first basis region and a second treatment image cropped on a basis of the second basis region to the classifier.

6. The information processing apparatus according to claim 5, wherein
the presentation unit presents, to the user, a graphical user interface (GUI) in which the first behavior information, the second behavior information, the first basis information, and the second basis information can be recognized.

7. The information processing apparatus according to claim 6, wherein
the input information includes at least one of a selection of the behavior information, a selection of the determination basis, an input of new behavior information different from the behavior information, an input of new basis information different from the basis information, or a new cropped treatment image different from the cropped treatment image.

8. The information processing apparatus according to claim 7, wherein
if the first behavior information or the second behavior information is correct, the storage unit stores the first behavior information or the second behavior information that is selected by the user via the GUI, as learning data of the plurality of recognizers and the classifier.

9. The information processing apparatus according to claim 7, wherein
if the first behavior information and the second behavior information are incorrect, the storage unit stores third behavior information as learning data of the plurality of recognizers and the classifier, the third behavior information being different from the first behavior information and the second behavior information that are input by the user via the GUI.

10. The information processing apparatus according to claim 7, wherein
if the first basis information or the second basis information is correct, the storage unit stores the first basis information or the second basis information that is selected by the user via the GUI, as learning data of the plurality of recognizers and the classifier.

11. The information processing apparatus according to claim 7, wherein
if the first behavior information and the second basis information are correct, the storage unit stores new basis information input by the user via the GUI, as learning data of the plurality of recognizers and the classifier.

12. The information processing apparatus according to claim 7, wherein
if the first basis information and the second basis information are incorrect, the storage unit stores third basis information as learning data of the plurality of recognizers and the classifier, the third basis information being different from the first basis information and the second basis information that are input by the user via the GUI.

13. An information processing method that is executed by a computer system, comprising:
acquiring a treatment image related to treatment;
outputting behavior information related to a behavior of a medical device related to the treatment, and a basis region indicating position information of a basis on which the behavior information is output, by inputting the treatment image to each of a plurality of recognizers;
outputting basis information related to the basis by inputting the treatment image cropped on the basis of the basis region to a classifier;
presenting a plurality of pieces of the behavior information and a plurality of pieces of the basis information to the user; and
storing input information input by the user on a basis of the behavior information and the basis information, as learning data of the plurality of recognizers and the classifier.

14. A program that causes a computer system to execute:
acquiring a treatment image related to treatment;
outputting behavior information related to a behavior of a medical device related to the treatment, and a basis region indicating position information of a basis on which the behavior information is output, by inputting the treatment image to each of a plurality of recognizers;
outputting basis information related to the basis by inputting the treatment image cropped on the basis of the basis region to a classifier;
presenting a plurality of pieces of the behavior information and a plurality of pieces of the basis information to the user; and
storing input information input by the user on a basis of the behavior information and the basis information, as learning data of the plurality of recognizers and the classifier.
